(19) United States
(12) Patent Application Publication
(10) Pub. No.: US 2026/0124336 A1
(43) Pub. Date: May 7, 2026

(54) ULTRASONIC ATOMIZER THAT CAN EASILY HOLD MULTIPLE FRAGRANCE LIQUID BOTTLES

(71) Applicant: DONGGUAN YIH TEH ELECTRIC PRODUCTS CO., LTD., DongGuan City (CN)

(72) Inventor: Ming Jen Hsiao, Miaoli County (TW)

(21) Appl. No.: 18/937,160

(22) Filed: Nov. 5, 2024

(51) Int. Cl. A61L 9/14; B05B 17/00; B05B 17/06

(52) U.S. Cl. CPC A61L 9/14; B05B 17/0646; B05B 17/0653; A61L 2209/132; A61L 2209/133

(57) ABSTRACT

The ultrasonic atomizer of the present invention is easy to mount multiple fragrance liquid bottles. The spray hole, atomizing device, upper assembly hole of the rack and elastic clip are respectively arranged in a plurality of corresponding groups in the atomizer to detachably mount multiple fragrance liquid bottles. This allows the atomizer to atomize the selected aroma liquid by controlling the atomizer function selection key of the atomizer control circuit, the corresponding aroma liquid bottle and the atomizing device. You can choose to change the fragrance at any time, with multiple fragrance selection functions. The liquid bottle can be easily pushed out of the ultrasonic atomizer and replaced with a new one conveniently and quickly, and then snapped into the atomizer for use.

### BACKGROUND OF THE INVENTION

### 1. Field of the Invention

The present invention relates to an atomizing device, and more particularly to an ultrasonic atomizer which is easy to hold a plurality of fragrance liquid bottles.

### 2. Description of the Related Art

There are some problems with replacing the fragrance liquid bottle used in the fragrance atomizer. The user's hand takes the fragrance liquid bottle and assembles or removes it from the frame inside the atomizer, and the hand and the fragrance liquid bottle are easily impacted or interfered by the shell around the atomizer, or the fragrance liquid bottle is difficult to assemble on the frame, and the fragrance liquid bottle is difficult to be pushed out of the atomizer, or the shell of the atomizer is inconvenient and time-consuming to open, making the replacement of the fragrance liquid bottle slow and inconvenient, for example, U.S. patent application Ser. No. 18/402,376, No. 16/259,654, No. 15/949,688, No. 16/162,364, No. 16/162,374, and No. 15/362,273.

In addition, the above-mentioned known fragrance atomizer can only produce one fragrance, and consumers cannot choose to change the fragrance.

### SUMMARY OF THE INVENTION

In view of the above problems of the prior art, the present invention provides an ultrasonic atomizer that is easy to mount multiple fragrance liquid bottles. The fragrance liquid bottles can be easily mounted or pushed out of the atomizer, and new fragrance liquid bottles can be easily and quickly replaced and then assembled in the atomizer for use.

The present invention also provides an ultrasonic atomizer that is easy to mount multiple fragrance liquid bottles and has a function of selecting multiple fragrances.

The present invention further provides an ultrasonic atomizer that is easy to mount multiple fragrance liquid bottles, and the fragrance liquid can be continuously supplied to the ultrasonic vibrator to effectively atomize and generate fragrance molecules for dispersion.

In some embodiments of the present invention, the ultrasonic atomizer that can be easily mounted with multiple fragrance liquid bottles comprises a housing, a rack, a control circuit, an electrical connection unit, and an atomizing device. The housing is a hollow housing, which comprises a top surface, a bottom surface, two trapezoidal side walls on both sides, a first opening, a second opening, and a spray hole. The space enclosed by the top surface, the bottom surface and the trapezoidal side walls on both sides forms the first opening on one side and the second opening on the other side. The trapezoidal side walls on both sides have side wall front edges, which extend downward and inwardly from both sides of the top surface along both sides of the second opening to the bottom surface in a gradually contracting manner, so that the area of the top surface is larger than the area of the bottom surface. A portion of the top surface beyond the front end of the bottom surface below the top surface is defined as a front top surface, and the spray hole is opened at the front top surface. The rack is coupled to the inner side of the housing. The rack comprises an upper assembly hole and an elastic clip facing the second opening. The atomizing device is combined with the upper side of the upper assembly hole and corresponds to the lower side of the spray hole, whereby the position of the upper assembly hole and the elastic clip has exceeded or at least corresponds to the front end of the bottom surface. The control circuit and the electrical connection unit are mounted in the first opening. The electrical connection unit is electrically connected to the control circuit and the control circuit is electrically connected to the atomizing device. Thereby, the elastic clip is used to detachably connect a liquid bottle. Thus, the elastic clip is used to detachably engage a liquid bottle. The position of the upper assembly hole and the elastic clip relative to the front end of the bottom surface has exceeded or at least corresponds to the front end of the bottom surface. When the user holds the liquid bottle and snaps it into or removes it from the elastic clip, the user is not easily disturbed by the second opening and can be easily snapped into or pushed out of the atomizer from the second opening. The user can conveniently and quickly replace a new liquid bottle and then assemble it in the atomizer for use.

The electrical connection unit is used to connect to a power supply unit to provide power to the atomizing device for generating ultrasonic waves to atomize the liquid, such as aromatic liquid or essential oil, provided in the liquid bottle.

In one embodiment of the present invention, multiple spray holes opened on the front top surface of the top surface, multiple atomizing devices, multiple upper assembly holes of the rack, and multiple elastic clips are respectively arranged in the atomizer for detachably combining a plurality of liquid bottles. The consumer can thereby control, for example, an atomizer function selection key electrically connected to the control circuit, and can continuously press the atomizer function selection key to correspond to different liquids filled in any of the liquid bottles, such as aromatic liquids or essential oils of different scents to generate scents through atomization, thereby achieving a variety of scent selection functions.

### BRIEF DESCRIPTION OF THE DRAWINGS

FIG. 1 is a three-dimensional external view of the ultrasonic atomizer of the present invention which is easy to mount multiple liquid bottles.

FIG. 2 is an exploded view of FIG. 1.

FIG. 3 is a side view of FIG. 1.

FIG. 4 is a side sectional view of FIG. 1.

FIG. 5 is a three-dimensional external view of the internal rack and the liquid bottle of the atomizer of the present invention.

FIG. 6 is a diagram showing a state in which the atomizing device and the liquid bottle can be freely detachably assembled in the ultrasonic atomizer.

FIG. 7 is a front cross-sectional view of FIG. 1.

FIG. 8 is another exploded view of FIG. 1.

FIG. 9 is a rear elevational view of FIG. 1.

### DETAILED DESCRIPTION OF THE PREFERRED EMBODIMENT

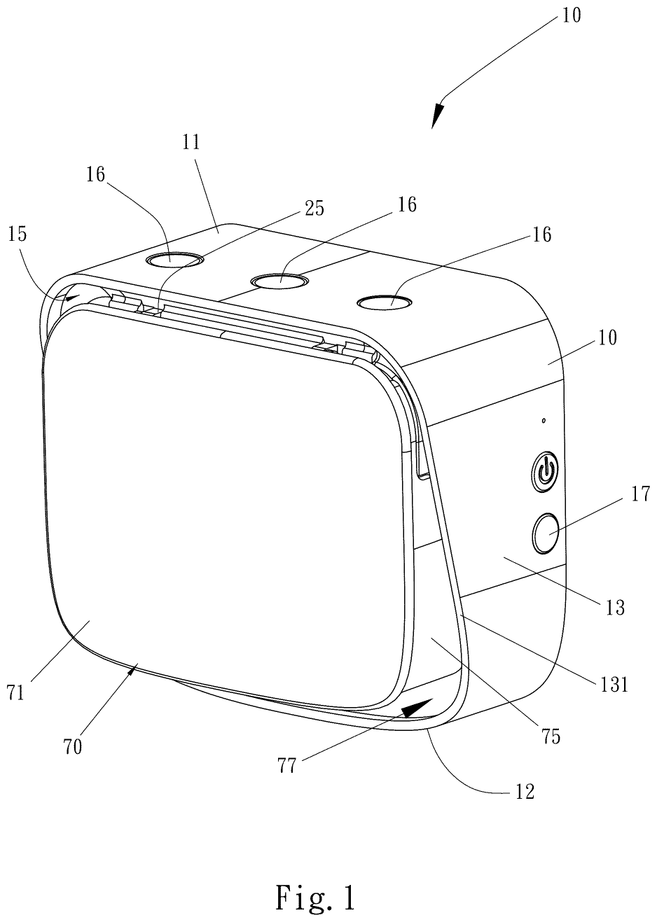

Please refer to FIGS. 1 to 5, some embodiments of an ultrasonic atomizer that is easy to mount multiple fragrance liquid bottles according to the present invention are shown. The atomizer comprises a housing 10, a rack 20, a control circuit 30, an electrical connection unit 40, and an atomizing device 50.

The housing 10 is a hollow housing, having a top surface 11, a bottom surface 12, trapezoidal side walls 13 on both sides, a first opening 14, a second opening 15, and a spray hole 16.

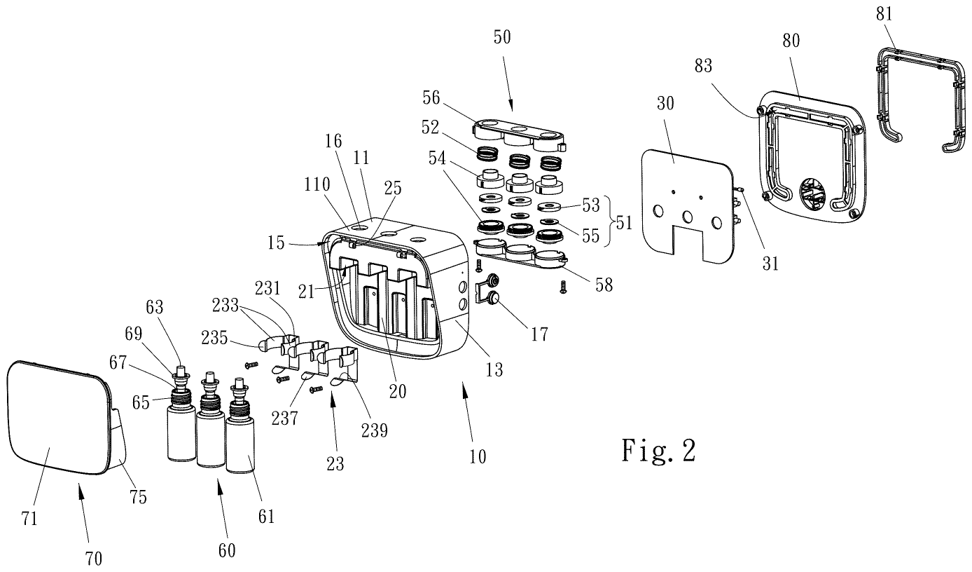

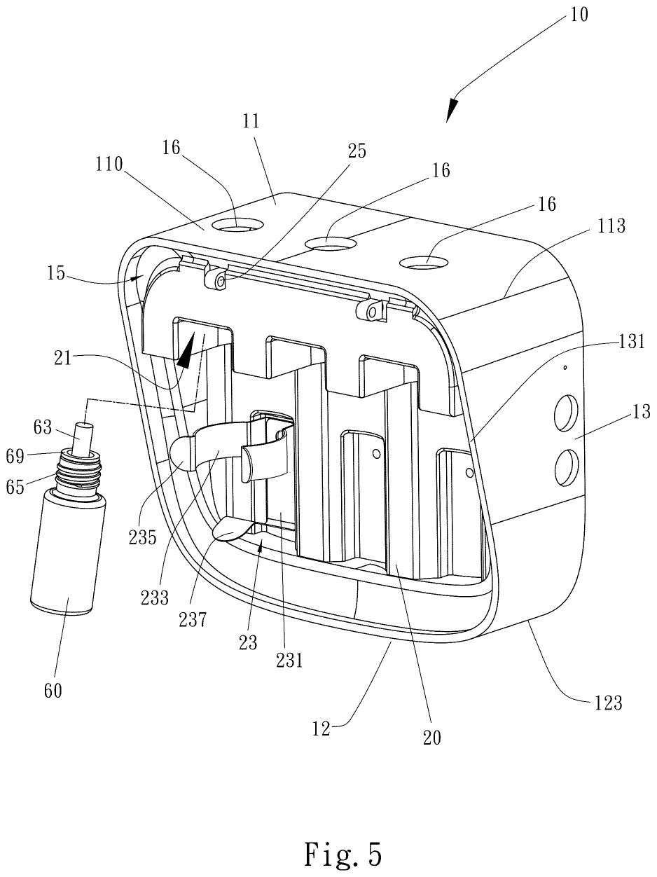

The space enclosed by the top surface 11, the bottom surface 12 and the trapezoidal side walls 13 on both sides forms the first opening 14 on one side and the second opening 15 on the other side. The trapezoidal side walls 13 on both sides have side wall front edges 131, which extend downward and inwardly from both sides of the top surface 11 along both sides of the second opening 15 to the bottom surface 12 in a gradually contracting manner, so that the area of the top surface 11 is larger than the area of the bottom surface 12. A portion of the top surface 11 beyond the front end 121 of the bottom surface 12 below the top surface 11 is defined as a front top surface 110 (see FIG. 2), and the spray hole 16 is opened at the front top surface 110 (see FIG. 5).

The rack 20 is coupled to the inner side of the housing 10. The rack 20 comprises an upper assembly hole 21 and an elastic clip 23 facing the second opening 15.

The atomizing device 50 is combined with the upper side of the upper assembly hole 21 and corresponds to the lower side of the spray hole 16, whereby the position of the upper assembly hole 21 and the elastic clip 23 has exceeded or at least corresponds to the front end 121 of the bottom surface 12.

The control circuit 30 and the electrical connection unit 40 are mounted in the first opening 14. The electrical connection unit 40 is electrically connected to the control circuit 30 and the control circuit 30 is electrically connected to the atomizing device 50.

Thus, the elastic clip 23 is used to detachably engage a liquid bottle 60, such as an essential oil bottle, a fragrance liquid bottle, a deodorant liquid bottle, or bottled water, to atomize the liquid. The position of the upper assembly hole 21 and the elastic clip 23 relative to the front end 121 of the bottom surface 12 has exceeded or at least corresponds to the front end 121 of the bottom surface 12. The user holds the liquid bottle 60 and snaps it onto or removes it from the elastic clip 23 without being easily impacted or disturbed by the side wall front edges 131 of the trapezoidal side walls 13 on both sides, the front end 121 of the bottom surface 12 and the rack 20. The side of the mouth 61 of the liquid bottle 60 can be easily assembled from bottom to top corresponding to the upper assembly hole 21, and the bottom of the liquid bottle 60 is not easily impacted by the front end 121 of the bottom surface 12. Furthermore, the liquid bottle 60 can be easily inserted or pushed out of the atomizer from the second opening 15 without being disturbed by the surroundings of the second opening 15, and a new liquid bottle 60 can be conveniently and quickly replaced and assembled in the atomizer for use.

The electrical connection unit 40 is used to connect to a power supply unit (such as the electrical plug in the figure, or a USB plug, or a battery, etc.) to provide power to the atomizing device 50 for generating ultrasonic waves to atomize the liquid filled in the liquid bottle 60, such as a scented liquid or essential oil, to generate fragrance in the environment.

It is to be noted that the term “the position has exceeded or at least corresponds to” should be understood in a broad sense, including but not limited to, for example, as shown in the embodiment diagram, the upper assembly hole 21 and the elastic clip 23 may be positioned beyond the front end 121 of the bottom surface 12, or correspond to the front end 121 of the bottom surface 12 below, or correspond to a short distance behind the 121 of the bottom surface 12 below, such as 0.1 mm to 30 mm. The meaning of the term can be understood by a person having ordinary knowledge in the relevant field.

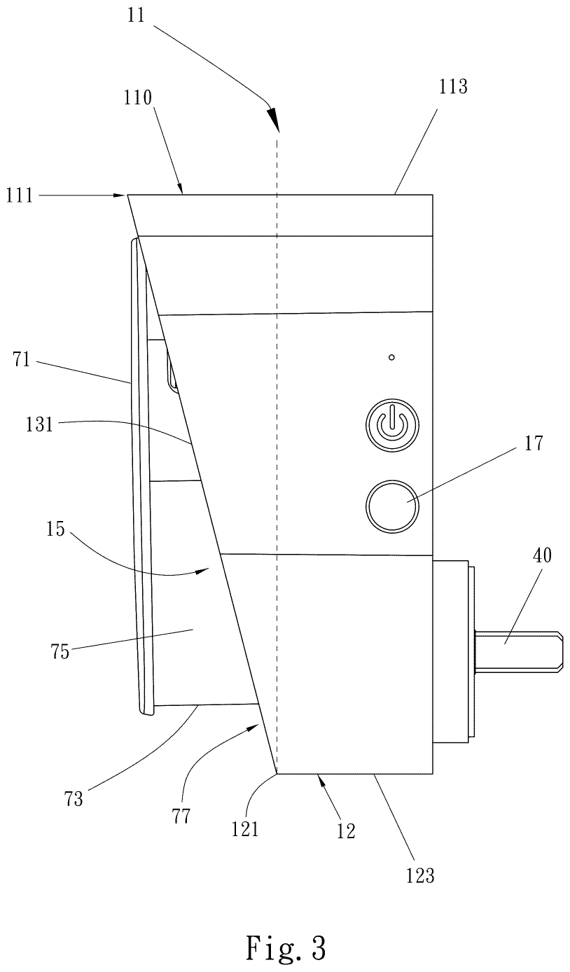

Referring to FIG. 1, FIG. 3 to FIG. 6. In one embodiment of the ultrasonic atomizer of the present invention, multiple spray holes 16 opened on the front top surface 110 of the top surface 11, multiple atomizing devices 50, multiple upper assembly holes 21 of the rack 20 and multiple elastic clips 23 are respectively arranged in in the atomizer for detachably combining a plurality of liquid bottles 60. In the embodiment, three are shown, through which the consumer can control the atomizer function selection key 17 electrically connected to the control circuit 30, and can continuously press the function selection key 17 to correspond to different liquids filled in any of the liquid bottles 60, such as atomizing aromatic liquids with different fragrances to produce fragrance, thereby achieving a variety of fragrance selection functions.

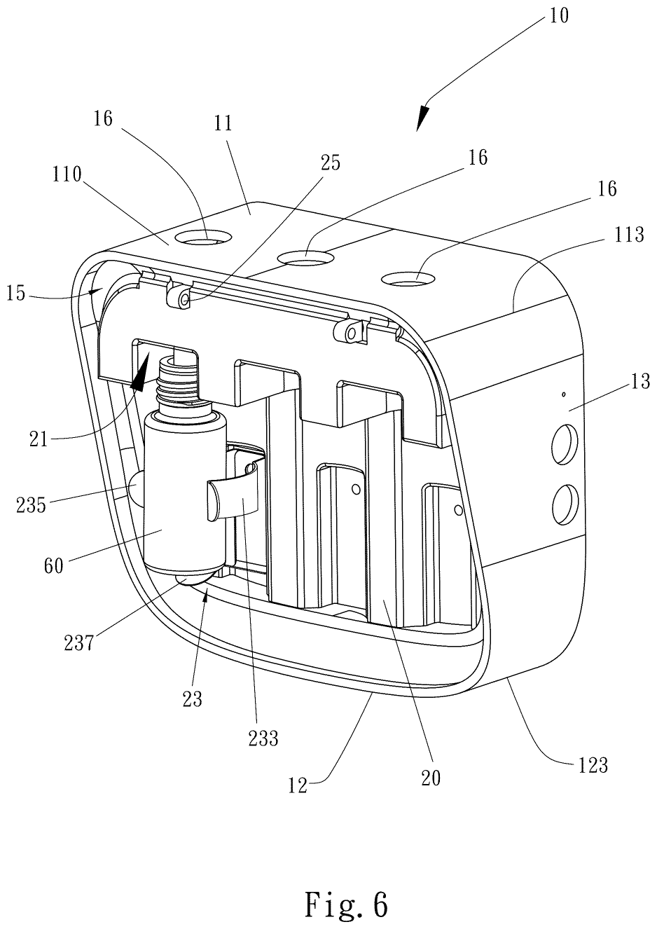

Referring to FIG. 5 and FIG. 6. In one embodiment of the ultrasonic atomizer of the present invention, the elastic clip 23 comprises a clip base 231 and a pair of circularly curved arms 233. The pair of circularly curved arms 233 protrude from both sides of the clip base 231. A gap is left between the two free ends of the pair of circularly curved arms 233 for inserting a liquid bottle and clamping it on the elastic clip 23. The diameter of the pair of circularly curved arms 233 and/or the distance between the two free ends may be slightly smaller than the diameter of the liquid bottle 60, so as to prepair the elasticity of the two circular arms 233 in clamping the liquid bottle 60. The free ends of the pair of circularly curved arms 233 are respectively extended with guide pieces 235, and the guide pieces 235 are turned outward from the free ends of the pair of circularly curved arms 233 to allow the liquid bottle 60 to be easily inserted between the pair of circularly curved arms 233 and to be temporarily fixed in the pair of circularly curved arms 233 in a detachable manner.

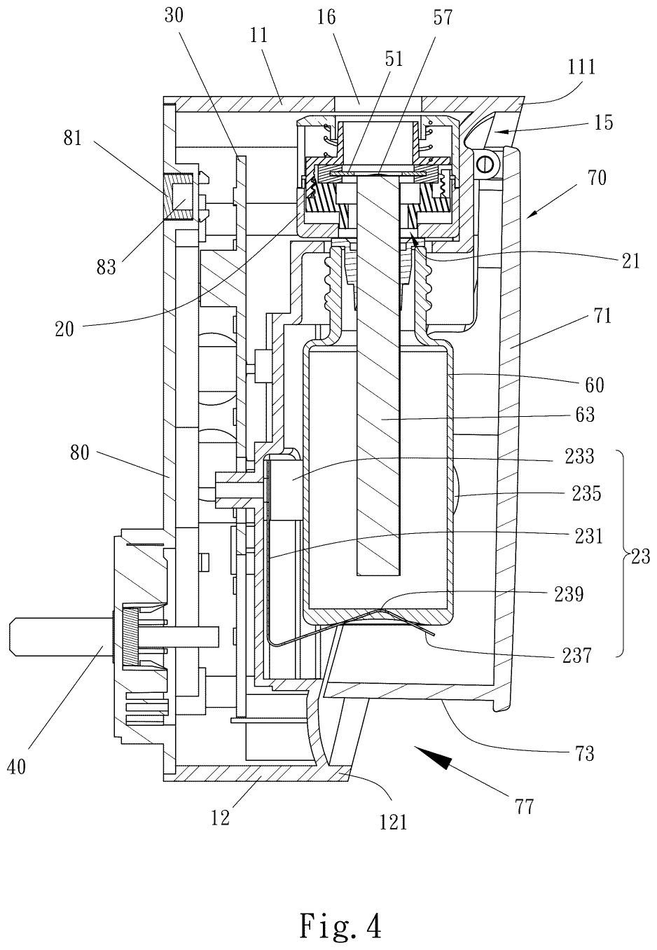

Please refer to FIGS. 4 to 6. In one embodiment of the ultrasonic atomizer of the present invention, the elastic clip 23 further comprises a spring 237, the spring 237 extends from the clip base 231 and is bent upward at an angle, the spring 237 is bent upward from the lower side of the clip base 231 for a distance, and one side of the free end of the spring 237 is further bent downward, and an elastic abutment portion 239 is formed at the bend.

Please refer to FIGS. 3 to 6. In one embodiment of the ultrasonic atomizer of the present invention, the top surface 11 and the bottom surface 12 are rectangular and respectively connect the trapezoidal side walls 13 on both sides, so that the length of the top surface short side 113 of the top surface 11 is greater than the length of the bottom surface short side 123 of the bottom surface 12. Thus, the area of the front edge of the top surface 11 that has exceeded the bottom surface short side 123 is the front top surface 110.

The atomizing device 50, the upper assembly hole 21 and the elastic clip 23 are arranged below the spray hole 16 of the front top surface 11, and extend beyond or at least correspond to the front edge of the bottom surface short side 123. As stated above, it is convenient for the user to pick up the liquid bottle 60 and attach or detach it inside the atomizer. The liquid bottle can be easily attached or pushed out of the atomizer through the second opening 15, and a new liquid bottle can be conveniently and quickly replaced and then assembled in the atomizer for use.

Please refer to FIGS. 3 to 6. In one embodiment of the ultrasonic atomizer of the present invention, the two sides of the second opening 15 are inclined upward and outwardly extended from the bottom surface front end 121 to the front edge of the front top surface 110. The inclined two sides of the second opening 15 are connected between the bottom surface front end 121 and the front edge of the front top surface 110 to form an inclined opening structure protruding beyond the bottom surface front end 121. In this way, the user can take the liquid bottle 60 from below the front top surface 110 and assemble or disassemble it on the rack 20 inside the housing 10. The hand and the liquid bottle 60 are not easily impacted or disturbed by the housing 10 and the rack 20 around the second opening 15, so that the liquid bottle 60 can be replaced easily and smoothly.

Please refer to FIGS. 1 to 4 and 8. An embodiment of the ultrasonic atomizer of the present invention further comprises a front cover 70. The upper side of the front cover 70 is hinged to the upper side of the rack 20, whereby the front cover 70 can be opened and closed by tilting up and down relative to the second opening 15, so as to easily cover the second opening 15 and the liquid bottle 60 therein. The front cover 70 can be opened relatively quickly to conveniently replace the liquid bottle 60 inside the atomizer, thereby solving the problem that the conventional atomizer is slow and inconvenient to open the cover, which is not conducive to replacing the liquid bottle in the atomizer.

The front cover 70 comprises a front plate 71, a bottom plate 73 horizontally extending a distance from the bottom edge of the front plate 71, and two triangular side plates 75 extending obliquely downward from both sides of the upper side of the front plate 71 to both sides of the bottom plate 73. The front plate 71 has a shaft 711 on its upper edge, and the rack 20 has a shaft hole 25 on its upper edge. The shaft 711 is rotatably inserted into the shaft hole 25. The front cover 70 and the rack 20 are thereby hinged, and the front cover 70 can be rotated relative to the second opening 15 to be opened, or returned to cover the second opening 15.

Please refer to FIGS. 1 to 4. In one embodiment of the ultrasonic atomizer of the present invention, a gap is predetermined between the front cover 70 and the trapezoidal side walls 13 and the bottom surface 12 on both sides of the housing 10, which is defined as an operating gap 77. The operating gap 77 is used to provide a user with a finger operating space, so that the user can insert the finger into the operating gap 77 to open the front cover 70 from the second opening 15 or to return the front cover 70 to cover the second opening 15.

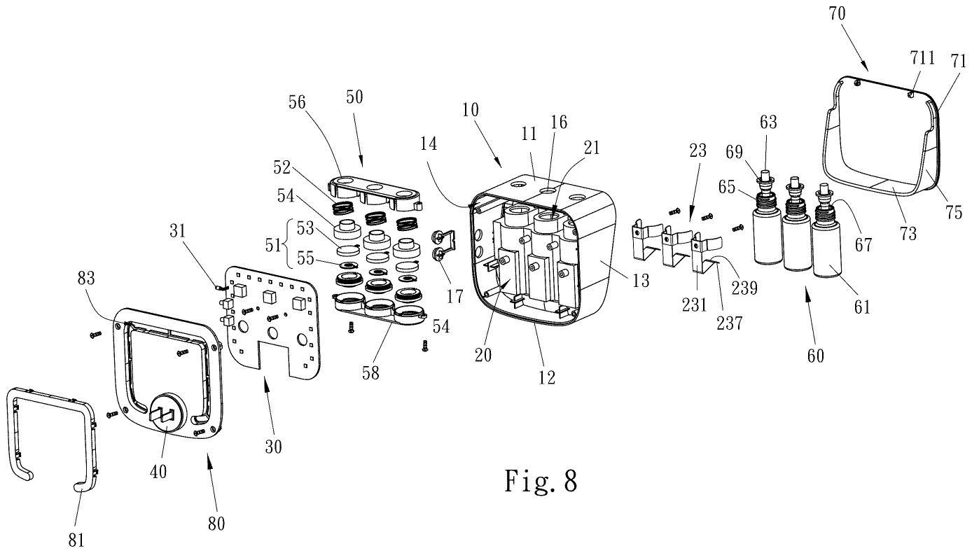

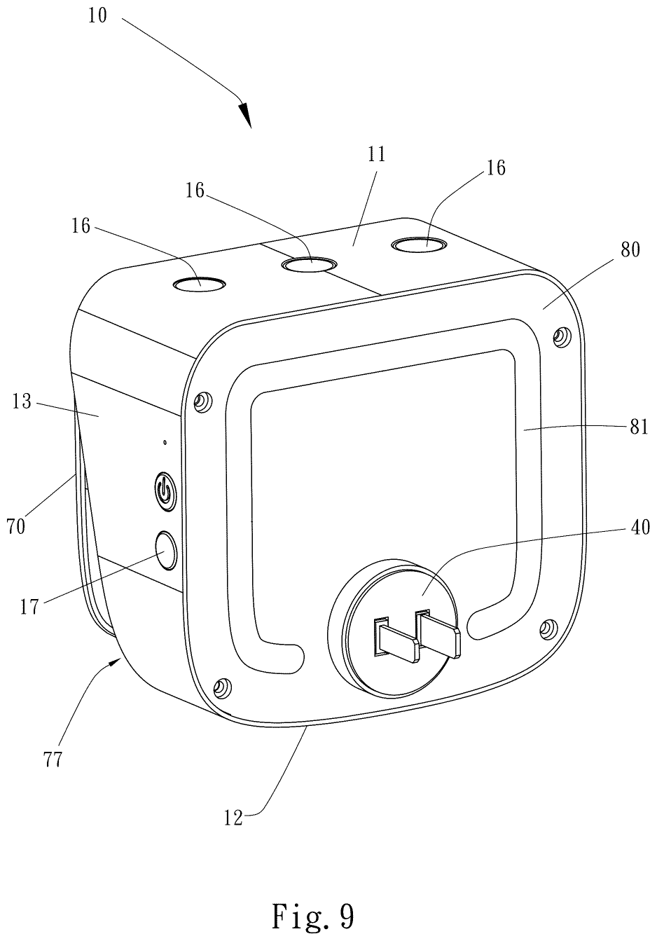

Please refer to FIGS. 8 and 9. In one embodiment of the present invention, the ultrasonic atomizer further comprises a rear cover 80 and a light-transmitting member 81. The rear cover 80 is provided with a recessed portion 83 corresponding to the light-transmitting member 81. The control circuit 30 comprises an electrically connected light emitting element 31. The rear cover 80 is coupled to the first opening 14, the light-transmitting member 81 is coupled to the recessed portion 83, and the light emitting element 31 corresponds to the inner side of the light-transmitting member 81. The electrical connection unit 40 can be connected to a power source to the control circuit 30 to provide power to the light emitting element 31, which can be any one of an LED, a light bulb, a halogen lamp, etc. In the embodiment, LED emits light to the light-transmitting member 81 to produce light decoration or lighting effect. The light-transmitting member 81 is made of any one of silicone, plastic, acrylic or glass.

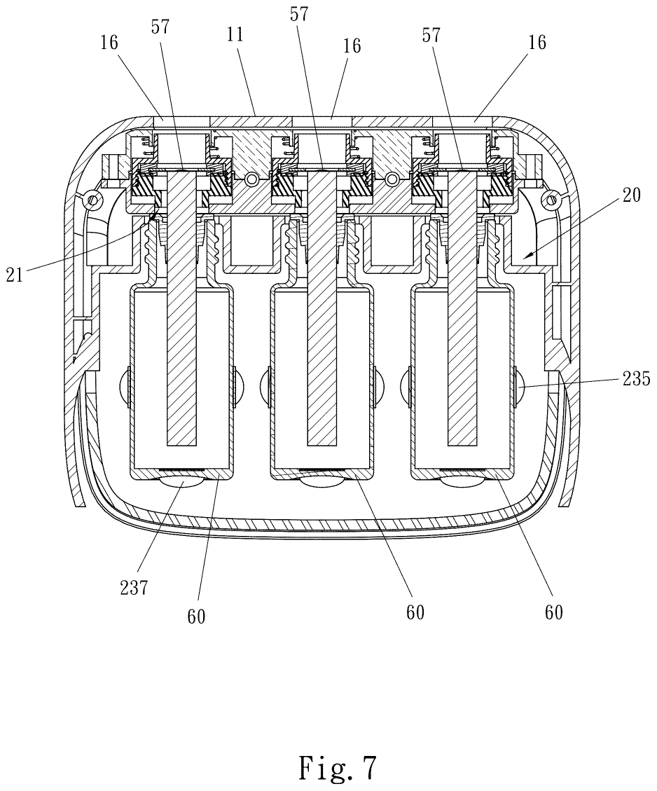

Please refer to FIGS. 4 to 8. In one embodiment of the ultrasonic atomizer of the present invention, the liquid bottle 60 comprises a bottle body 61, a water-conducting core 63, a neck 65, a mouth 67, a liquid (not shown), and a through bottle stopper 69. The liquid is filled in the bottle body 61. The neck 65 extends from the upper side of the bottle body 61. The mouth 57 is formed at the upper end of the neck 65. One side of the water-conducting core 63 is engaged in the mouth 67 to contact the liquid, and the other side of the water-conducting core 63 protrudes out of the mouth 67.

Please refer to FIGS. 4 to 8. In one embodiment of the ultrasonic atomizer of the present invention, the atomizing device 50 comprises a piezoelectric vibrating disc 51, an elastic member 52, a center-opened casing 54, a center-opened upper cover 56, and a center-opened lower cover 58. The piezoelectric vibrating disc 51 comprises a ring-shaped piezoelectric ceramic plate 53 and a conical vibrating plate 55. In the embodiment, the conical vibrating plate 55, which is made of nickel alloy or iron alloy, has a circular center with a multi-porous convex portion 57. The opening of the ring-shaped piezoelectric ceramic plate 53 may be penetrated at the periphery of the multi-porous convex portion 57, so that the multi-porous convex portion 57 is assembled in the opening of the ring-shaped piezoelectric ceramic plate 53 to form the piezoelectric vibrating disc 51. The piezoelectric vibrating disc 51 is coupled to the center-opened casing 54, the center-opened casing 54 is coupled between the center-opened upper cover 56 and the center-opened lower cover 58, and the elastic member 52 is coupled between the center-opened upper cover 56 and the center-opened casing 54 in a compression-accumulated tension manner.

The mouth 61 of the liquid bottle 60 is assembled in the upper assembly hole 21. The top of the free end of the water-conducting core 63 passes through the center-opened casing 54 and abuts against the lower side of the multi-porous convex portion 57. The accumulated tension of the elastic member 52 pushes against the center-opened casing 54 and links the multi-porous convex portion 57 inside it, and continuously abuts against the liquid (such as aromatic liquid) at the free end of the water-conducting core 63. The power source connected to the electrical connection unit 40 is provided to the control circuit 30 and the piezoelectric vibrating disc 51 to generate ultrasonic vibrations, so as to continuously and effectively contact the top liquid of the free end of the water-conducting core 63, such as essential oil, to atomize and produce, for example, fragrance or deodorizing gas or atomized water droplets.

The water-conducting core 63 is, for example, a water-conducting fiber rod, which is made of, for example, PET, PP, TPU or TPEE and has good porosity to form a better water-conducting and water-absorbing capillary effect. The water-conducting fiber rod absorbs the liquid (aromatic liquid or essential oil) in the liquid bottle 60 and can transfer an appropriate amount of the aromatic liquid to its free end. The free end of the water-conducting fiber rod will not have excessive aromatic liquid flowing back or accumulating in the multi-porous convex portion 57, and the free end of the water-conducting core 63 of the fiber rod is elastic and will not bend or deflect to cause looseness. Liquid such as essential oil or aromatic liquid that continuously contacts the multi porous convex portion 57 is vibrated and continuously and effectively atomized into aromatic liquid molecules, which are dispersed into the space through the multi-porous convex portion 57, the center-opened casing 54, and the center-opened upper cover 56 to generate fragrance.

What the invention claimed is:

1. An ultrasonic atomizer, comprising: a housing, a rack, a control circuit, an electrical connection unit, and at least one atomizing device, wherein:
said housing is a hollow housing, comprising a top surface, a bottom surface, two trapezoidal side walls on two opposite sides, a first opening, a second opening, and at least one spray hole, said trapezoidal side walls being located on two opposite sides of said top surface and extending downward to said bottom surface, said top surface, said bottom surface and said trapezoidal side walls enclosing a space with said first opening first opening formed on one side and said second opening formed on an opposite side, said two trapezoidal side walls having side wall front edges thereof respectively extending downward and inwardly from two opposite sides of said top surface along two opposite sides of said second opening to said bottom surface in a gradually contracting manner, so that the area of said top surface is larger than the area of said bottom surface, said top surface having an area thereof exceeding a front end of said bottom surface below and defined as a front top surface, said spray hole being opened on said front top surface;
said rack is coupled to an inner side of said housing, and said rack comprises at least one upper assembly hole on an upper side thereof and at least one elastic clip on a lower side thereof facing said second opening;
said at least one atomizing device is respectively combined with said at least one upper assembly hole at an upper side and corresponds to a lower side of said at least one spray hole, whereby said at least one upper assembly hole and said at least one elastic clip are positioned beyond or at least correspond to the said front end of said bottom surface;
said control circuit and said electrical connection unit are joined in said first opening, said electrical connection unit being electrically connected to said control circuit and said control circuit being electrically connected to said atomizing device.

2. The ultrasonic atomizer as claimed in claim 1, wherein the number of said at least one spray hole opened on said front top surface of said top surface, the number of said at least one atomizing device, the number of said at least one upper assembly hole of said rack and the number of said at least one elastic clip are multiple and respectively arranged in the ultrasonic atomizer for detachably combining a plurality of liquid bottles.

3. The ultrasonic atomizer as claimed in claim 1, wherein each said elastic clip comprises a clip base, a pair of circularly curved arms protruded from two opposite sides of said clip base and a gap left between two free ends of said pair of circularly curved arms for inserting a liquid bottle and clamping the liquid bottle on said elastic clip, the diameter of said pair of circularly curved arms and/or the distance between the two free ends of said pair of circularly curved arms being smaller than the diameter of the liquid bottle to provide the elasticity of said two circularly curved arms in clamping the liquid bottle, each said circularly curved arm having a guide piece extended from the free end thereof, said guide piece being turned outward from the free end of the respective said circularly curved arm.

4. The ultrasonic atomizer as claimed in claim 3, wherein each said elastic clip further comprises a spring bent upward from a lower side of said clip base for a distance, said spring having a free end thereof bent downward so that an elastic abutment portion is formed at the bending position.

5. The ultrasonic atomizer as claimed in claim 1, wherein said top surface and said bottom surface are rectangular and respectively connect said trapezoidal side walls on two opposite sides, so that the length of the short side of said top surface is greater than the length of the short side of said bottom surface, whereby the area of a front edge of said top surface that has exceeded the short side of said bottom surface is the said front top surface.

6. The ultrasonic atomizer as claimed in claim 5, wherein said at least one spray hole, said at least one atomizing device, said at least one upper assembly hole and said at least one elastic clip are located so as to protrude beyond or at least correspond to the front edge of the short side of said bottom surface.

7. The ultrasonic atomizer as claimed in claim 6, wherein said second opening has two sides thereof inclined upward and outwardly extended from the front end of said bottom surface to the front edge of said front top surface, and the two inclined sides of said second opening are connected between the front end of said bottom surface and the front edge of said front top surface to form an inclined opening structure protruding beyond the front end of said bottom surface.

8. The ultrasonic atomizer as claimed in claim 7, further comprising a front cover, said front cover having an upper side thereof hinged to the upper side of said rack, so that said front cover is able to be opened and closed by tilting up and down relative to said second opening.

9. The ultrasonic atomizer as claimed in claim 8, wherein said rack further comprises a shaft hole located at an upper edge thereof; said front cover comprises a front plate, a bottom plate extending horizontally from a bottom edge of said front plate and two triangular side plates extending downward from two opposite sides of a top of said front plate to two opposite sides of said bottom plate, said front plate being provided with a shaft at an upper edge thereof, said shaft being rotatably inserted into said shaft hole of said rack to form a hinge, so that said front cover is rotatable relative to said second opening to be opened, or returned to cover said second opening.

10. The ultrasonic atomizer as claimed in claim 9, wherein said front cover and said trapezoidal side walls and said bottom surface of said housing define an operating gap therebetween.

11. The ultrasonic atomizer as claimed in claim 10, further comprising a rear cover and a light-transmitting member, said rear cover being coupled to said first opening and provided with a recessed portion corresponding to said light-transmitting member, said light-transmitting member being combined with said recessed portion; said control circuit comprises an electrically connected light emitting element located corresponding to an inner side of said light-transmitting member.

12. The ultrasonic atomizer as claimed in claim 1, further comprising at least one liquid bottle detachably mounted in said rack, each said liquid bottle comprising a bottle body, a water-conducting core, a neck, a mouth, a liquid and a through bottle stopper, said liquid being filled in said liquid bottle, said neck extending from an upper side of said bottle body, said mouth being formed at an upper end of said neck, said water-conducting core having one side thereof engaged in said mouth to contact said liquid and an opposite side thereof protruded out of said mouth.

13. The ultrasonic atomizer as claimed in claim 1, wherein each said atomizing device comprises a piezoelectric vibrating disc, an elastic member, a center-opened casing, a center-opened upper cover and a center-opened lower cover, said piezoelectric vibrating disc being composed of a ring-shaped piezoelectric ceramic plate and a conical vibrating plate, said conical vibrating plate comprising a multi-porous convex portion in the center thereof, said ring-shaped piezoelectric ceramic plate being attached onto the periphery of said multi-porous convex portion so that said multi-porous convex portion is located in the center of said ring-shaped piezoelectric ceramic plate to form said piezoelectric vibrating disc, said piezoelectric vibrating disc being coupled to said center-opened casing, said center-opened casing being coupled between said center-opened upper cover and said center-opened lower cover, said elastic member being compressed and tension-accumulated and being coupled between said center-opened upper cover and said center-opened casing.
